(19) United States
(12) Patent Application Publication
(10) Pub. No.: US 2017/0099875 A1
(43) Pub. Date: Apr. 13, 2017

(54) Systems and Methods for Resin Cleaning and Sterilizing

(71) Applicants: Daniel D. Voss, Hartford, WI (US); Ronald R. Burghardt, Redgranite, WI (US)

(72) Inventors: Daniel D. Voss, Hartford, WI (US); Ronald R. Burghardt, Redgranite, WI (US)

(21) Appl. No.: 15/164,351

(22) Filed: May 25, 2016

Related U.S. Application Data
(60) Provisional application No. 62/239,560, filed on Oct. 9, 2015.

(51) Int. Cl. A24F 9/04; B08B 3/08; A61L 2/18; B08B 9/023; B08B 9/032; B08B 3/12; B08B 3/10

(52) U.S. Cl. CPC A24F 9/04; B08B 3/12; B08B 3/08; B08B 3/10; B08B 9/023; B08B 9/032; A61L 2/18; A61L 2202/17; A61L 2202/11; A61L 2202/14

(57) ABSTRACT

Systems and methods for resin cleaning are disclosed. In particular, systems and methods are provided for a resin cleaning and sterilizing system that utilizes at least two ultrasonic cleaners. A first ultrasonic cleaner provides removal of resin on a smoking apparatus, and a second ultrasonic cleaner provides sterilization of the smoking apparatus.

### CROSS-REFERENCE TO RELATED APPLICATION

The present application is based on, claims priority to, and incorporates by reference in its entirety, U.S. Provisional Patent Application No. 62/239,560, filed Oct. 9, 2015, and entitled “Systems and Methods for Resin Cleaning and Sterilizing.”

### STATEMENT OF FEDERALLY SPONSORED RESEARCH OR DEVELOPMENT

Not applicable.

### TECHNICAL FIELD

This invention relates generally to cleaning systems and, more specifically, to systems and methods for cleaning a smoking apparatus at least partially covered with resin/tar.

### BACKGROUND

It is well known that use of a smoking apparatus (e.g., a pipe, a water pipe, or a tube) causes a build up of resin and tar on the smoking apparatus due to condensation of the combusted organic material (e.g., tobacco or medical marijuana) being smoked. This build up of resin may lead to the smoking apparatus not functioning properly, for example, due to blocked air flow passageways. Additionally, the resin typically contains harmful compounds which, when reignited and smoked, may pose a health risk. Furthermore, since the resin is mainly composed of organic material, it can be susceptible to molding and/or rotting over time.

Typically, a smoking apparatus is cleaned via a pipe cleaner and/or a cleaning solution. However, these cleaning methods rarely sufficiently clean (i.e., remove the resin) and sterilize the smoking apparatus.

Hence, a need exists for a resin cleaning and sterilizing system that efficiently and effectively cleans and sterilizes a smoking apparatus.

### SUMMARY

The present invention provides systems and methods for resin cleaning. In particular, systems and methods are provided for a resin cleaning and sterilizing system that utilizes at least two ultrasonic cleaners. A first ultrasonic cleaner provides removal of resin on a smoking apparatus, and a second ultrasonic cleaner provides sterilization of the smoking apparatus.

In one aspect, the present invention provides a resin cleaning and sterilizing system for a smoking apparatus. The resin cleaning and sterilizing system includes a first ultrasonic cleaner having a first cavity configured to be filled with a cleaning solution and a first mesh basket. The resin cleaning and sterilizing system further includes a second ultrasonic cleaner having a second cavity configured to be filled with a sterilizing solution and a second mesh basket. The first ultrasonic cleaner and the second ultrasonic cleaner are mounted on a mounting stand.

In another aspect, the present invention provides a method of cleaning and sterilizing a smoking apparatus using a resin cleaning and sterilizing system. The resin cleaning and sterilizing system includes a first ultrasonic cleaner having a first cavity configured to be filled with a cleaning solution and a first mesh basket, and a second ultrasonic cleaner having a second cavity configured to be filled with a sterilizing solution and a second mesh basket. The method includes submerging the smoking apparatus into the cleaning solution of the first ultrasonic cleaner, activating the first ultrasonic cleaner for a first predetermined length of time, and removing the smoking apparatus from the cleaning solution after the first predetermined length of time. The method further includes submerging the smoking apparatus into the sterilizing solution of the second ultrasonic cleaner, activating the second ultrasonic cleaner for a second predetermined length of time, and removing the smoking apparatus from the sterilizing solution after the second predetermined length of time.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE FIGURES

FIG. 1 is a perspective view of a disassembled resin cleaning and sterilizing system in accordance with one embodiment of the present invention.

FIG. 2 is a perspective view of the resin cleaning and sterilizing system of FIG. 1 assembled.

FIG. 3 is a perspective view of a basket divider of the resin cleaning and sterilizing system of FIG. 1.

FIG. 4 is a top view of the basket cleaner of FIG. 3 installed into a basket of the resin cleaning and sterilizing system of FIG. 1.

FIG. 5 outlines the steps for operating the resin cleaning and sterilizing system of FIG. 1 according to one aspect of the present invention.

### DETAILED DESCRIPTION

The use of the term “resin” herein is a term used to describe a tar-like substance that forms on a smoking apparatus (e.g., a pipe) due to condensation of the combusted organic material (e.g., tobacco or medical marijuana) being smoked.

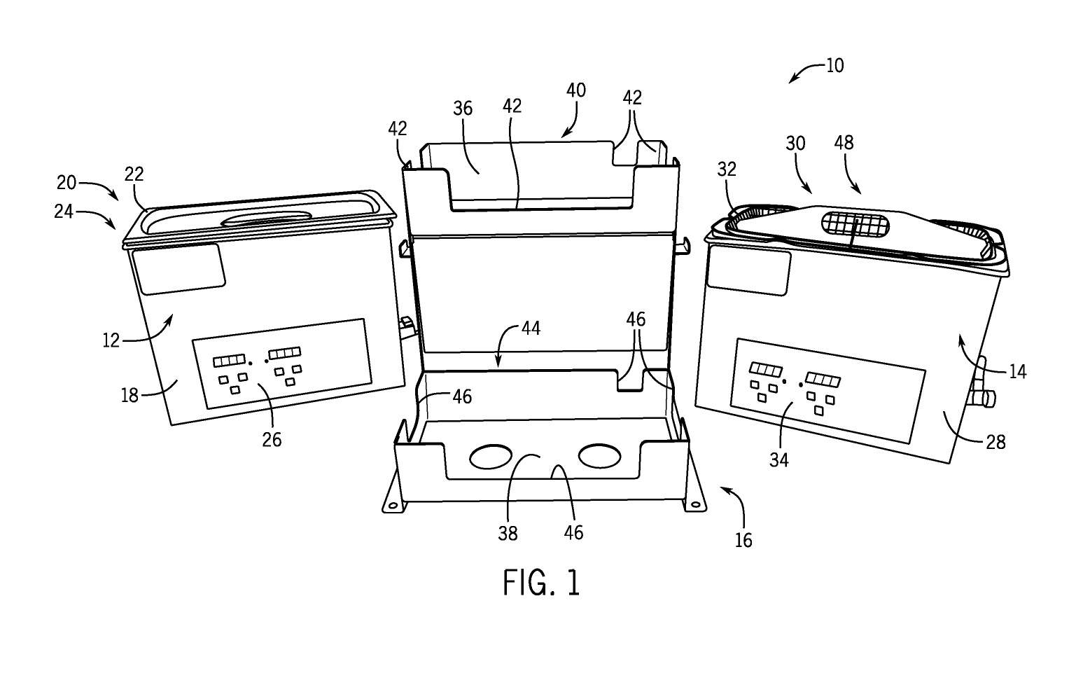

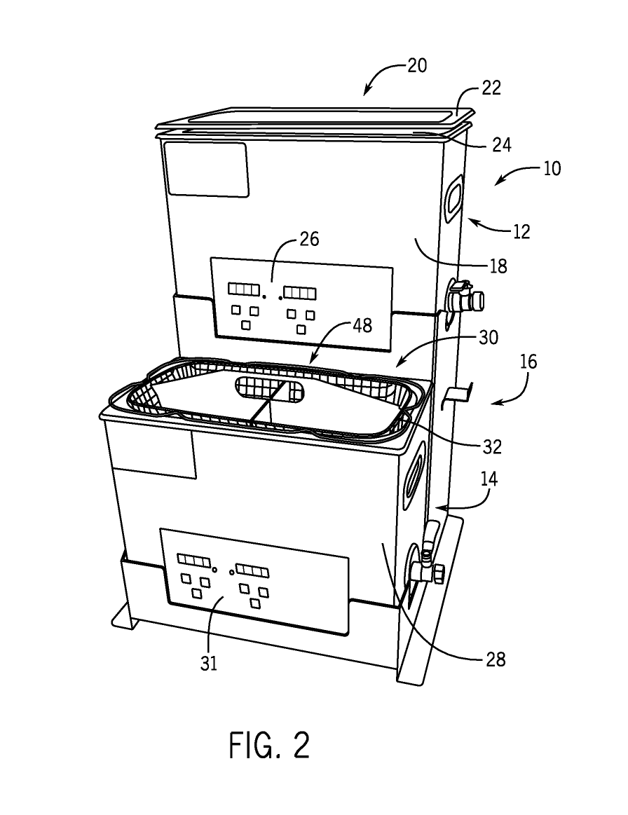

FIGS. 1 and 2 show a resin cleaning and sterilizing system 10 in accordance with one form of the present invention. The resin cleaning and sterilizing system 10 includes a first ultrasonic cleaner 12, a second ultrasonic cleaner 14, and a mounting stand 16. The first ultrasonic cleaner 12 and the second ultrasonic cleaner 14 are each configured to generate ultrasonic waves that travel through a fluid (i.e., an ultrasonic bath) within the respective ultrasonic cleaners 12 and 14 at a frequency generally between 20 kHz and 400 kHz. Ultrasonic waves in this frequency range generate cavitation bubbles which agitate the fluid within the ultrasonic cleaners 12 and 14, and enable the fluid to remove resin, or other debris, from a smoking apparatus and sterilize the smoking apparatus, as will be described in detail below.

The first ultrasonic cleaner 12 includes a first housing 18 defining a first cavity 20, and a first cover 22. The first cavity 20 can be filled with a cleaning solution (e.g., Greased-Lightning™ Cleaner and Degreaser) thereby forming a first ultrasonic bath. A first mesh basket 24 is dimensioned to be received within the first cavity 20. The first mesh basket 24 enables a user of the resin cleaning and sterilizing system 10 to easily place a smoking apparatus (not shown) into the first ultrasonic bath and subsequently remove the smoking apparatus from the first ultrasonic bath. The first cover 22 is dimensioned to cover the first cavity 20 and protect a user of the resin cleaning and sterilizing system 10 from splashing cleaning solution.

A first control panel 26 is arranged on the first housing 18 that enables a user of the resin cleaning and sterilizing system 10 to control, for example, a duration of time for the ultrasonic bath to be active (i.e., generate ultrasonic waves that travel through the first ultrasonic bath), a temperature of the cleaning solution in the first ultrasonic bath, and a frequency of the waves traveling through the first ultrasonic bath.

The second ultrasonic cleaner 14 includes a second housing 28 defining a second cavity 30, and a second cover (not shown). The second cavity 30 can be filled with a sterilizing solution (e.g., Steramine™ tablets mixed with water) thereby forming a second ultrasonic bath.

A second mesh basket 32 is dimensioned to be received within the second cavity 30. The second mesh basket 32 enables a user of the resin cleaning and sterilizing system 10 to easily place the smoking apparatus into the second ultrasonic bath and subsequently remove the smoking apparatus from the second ultrasonic bath. The second cover is dimensioned to cover the second cavity 30 and protect a user of the resin cleaning and sterilizing system 10 from splashing sterilizing solution.

A second control panel 34 is arranged on the second housing 28 that enables a user of the resin cleaning and sterilizing system 10 to control, for example, a duration of time for the second ultrasonic bath to be active (i.e., generate ultrasonic waves that travel through the second ultrasonic bath), a temperature of the cleaning solution in the second ultrasonic bath, and a frequency of the waves traveling through the second ultrasonic bath.

The mounting stand 16 includes a first platform 36 dimensioned to receive the first ultrasonic cleaner 12 and a second platform 38 dimensioned to receive the second ultrasonic cleaner 14. The first platform 36 includes a first supporting frame 40 which extends substantially perpendicularly from the first platform 38. The first supporting frame 40 aids in preventing the first ultrasonic cleaner 12 from shifting when placed upon the first platform 36. The first supporting frame 40 includes one or more cutouts 42 for providing a user of the resin cleaning and sterilizing system 10 access to the first control panel 26 and/or for receiving a drainage port and/or a power chord of the first ultrasonic cleaner 12. As shown in FIGS. 1 and 2, the first supporting frame 40 includes four cutouts 42. In other non-limiting examples, the first supporting frame 40 may include any number of cutouts 42, as desired.

The second platform 38 includes a second supporting frame 44 which extends substantially perpendicularly from the second platform 38. The second supporting frame 44 aids in preventing the second ultrasonic cleaner 14 from shifting when placed upon the second platform 38. The second supporting frame 44 includes one or more cutouts 46 for providing a user of the resin cleaning and sterilizing system 10 access to the second control panel 34 and/or for receiving a drainage port and/or a power chord of the second ultrasonic cleaner 14. As shown in FIGS. 1 and 2, the second supporting frame 44 includes four cutouts 46. In other non-limiting examples, the second supporting frame 44 may include any number of cutouts 46, as desired.

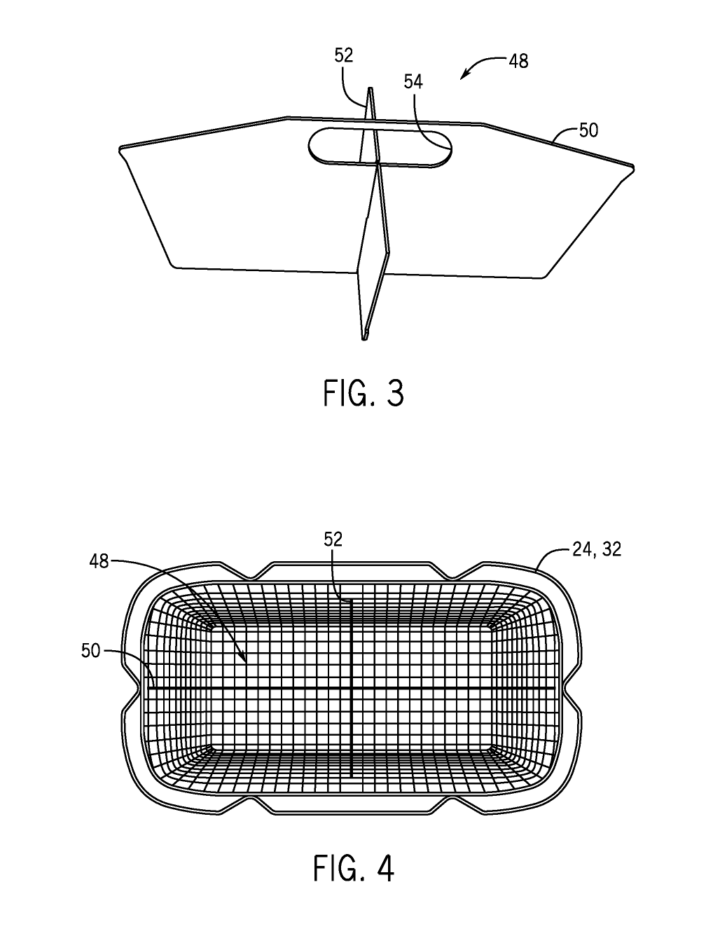

With reference to FIGS. 3 and 4, the first mesh basket 24 and/or the second mesh basket 32 can include a dividing member 48. The dividing member 48 includes a first plate 50 slidably coupled to a second plate 52. As shown in FIGS. 3 and 4, the first plate 50 is dimensioned to be received longitudinally within the first mesh basket 24 and/or the second mesh basket 32. The first plate 50 includes an aperture 54 that enables a user of the resin cleaning and sterilizing system 10 to grip the dividing member 48. The second plate 52 is dimensioned to be received latitudinally within the first mesh basket 24 and/or the second mesh basket 32.

The dividing member 48 enables a user to selectively divide the first mesh basket 24 and/or the second mesh basket 32 into one or more compartments. Specifically, when both the first plate 50 and the second plate 52 are installed, the dividing member 48 divides the first mesh basket 24 and/or the second mesh basket 32 into four compartments. When either the first plate 50 or the second plate 52 are installed, the dividing member 48 divides the first mesh basket 24 and/or the second mesh basket 32 into two compartments. It should be known that the specific arrangement of the dividing member 48 is not meant to be limiting in any way, and one of skill in the art would appreciate that the dividing member 48 may be altered to selectively divide the first mesh basket 24 and/or the second mesh basket 32 into any number of compartments, as desired.

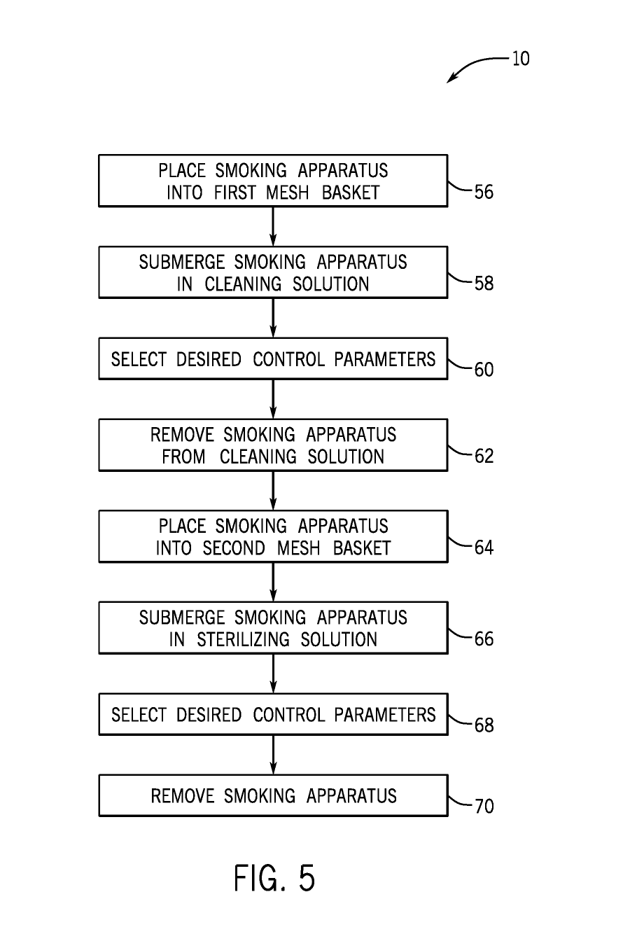

One non-limiting example of the operation of the resin cleaning and sterilizing system will be described with reference to FIGS. 1-5. A user begins at step 56 by placing a smoking apparatus at least partially covered with resin in the first mesh basket 24. One or more smoking apparatuses may be placed into the first mesh basket 24 if the dividing member 48 is installed. Next, at step 58, the user places the first mesh basket 24 into the first ultrasonic bath of the first ultrasonic cleaner 12, such that the smoking apparatus is submerged within the cleaning solution. The user then sets, at step 60, at least one of a desired length of time for the first ultrasonic bath to be active, a desired temperature of the first ultrasonic bath, and/or a frequency of the first ultrasonic bath via the first control panel 26. In one non-limiting example, the first ultrasonic bath may be set to between approximately 18° C. and 100° C. In another non-limiting example, the first ultrasonic bath may be set to between approximately 18° C. and 80° C. In yet another non-limiting example, the first ultrasonic bath may be set to greater than approximately 35° C.

Depending on the amount of resin on the smoking apparatus, there may be a predetermined amount of time for the first ultrasonic bath to be active in order to remove the resin. Once the smoking apparatus has been submerged in the cleaning solution of the first ultrasonic cleaner 12 for a predetermined amount of time, with the first ultrasonic cleaner active 12, the smoking apparatus will then be cleaned of resin and can be removed from the cleaning solution at step 62.

Following the smoking apparatus being removed from the cleaning solution of the first ultrasonic cleaner 12 at step 62, the smoking apparatus can then be placed, at step 64, into the second mesh basket 32. One or more smoking apparatuses may be placed into the second mesh basket 32 if the dividing member 48 is installed. Next, at step 66 the user places the second mesh basket into the second ultrasonic bath of the second ultrasonic cleaner, such that the smoking apparatus is submerged within the sterilizing solution. The user then sets, at step 68, at least one of a desired length of time for the second ultrasonic bath to be active, a desired temperature of the second ultrasonic bath, and/or a frequency of the second ultrasonic bath via the second control panel 34. In one non-limiting example, the second ultrasonic bath may be set to between approximately 18° C. and 100° C. In another non-limiting example, the second ultrasonic bath may be set to between approximately 18° C. and 80° C. In yet another non-limiting example, the second ultrasonic bath may be set to greater than approximately 35° C.

There may be a predetermined amount of time for the second ultrasonic bath to be active to ensure the smoking apparatus is sterilized. Once the smoking apparatus has been submerged in the sterilizing solution of the second ultrasonic cleaner 14 for a predetermined amount of time, with the second ultrasonic cleaner 14 active, the smoking apparatus will then be sterilized and is removed from the sterilizing solution at step 70.

Thus, as described above, the resin cleaning and sterilizing system 10 enables a user to clean the resin off and sterilize a smoking apparatus.

It should be appreciated that various other modifications and variations to the preferred embodiments can be made within the spirit and scope of the invention. Therefore, the invention should not be limited to the described embodiments. To ascertain the full scope of the invention, the following claims should be referenced.

What is claimed is:

1. A resin cleaning and sterilizing system for a smoking apparatus, comprising:
a first ultrasonic cleaner including a first cavity configured to be filled with a cleaning solution and to receive a first mesh basket;
a second ultrasonic cleaner including a second cavity configured to be filled with a sterilizing solution and to receive a second mesh basket; and
wherein the first ultrasonic cleaner and the second ultrasonic cleaner are mounted on a mounting stand, and are configured to clean the smoking apparatus of resin and sterilize the smoking apparatus.

2. The resin cleaning and sterilizing system of claim 1, wherein the first ultrasonic cleaner includes a first control panel.

3. The resin cleaning and sterilizing system of claim 2, wherein the first control panel is configured to control at least one of a time for the first ultrasonic cleaner to be active, a temperature of the cleaning solution within the first cavity, and a frequency output by the first ultrasonic cleaner.

4. The resin cleaning and sterilizing system of claim 1, wherein the second ultrasonic cleaner includes a second control panel.

5. The resin cleaning and sterilizing system of claim 4, wherein the second control panel is configured to control at least one of a time for the second ultrasonic cleaner to be active, a temperature of the sterilizing solution within the second cavity, and a frequency output by the second ultrasonic cleaner.

6. The resin cleaning and sterilizing system of claim 1, wherein the first mesh basket includes a dividing member.

7. The resin cleaning and sterilizing system of claim 6, wherein the dividing member selectively divides the first mesh basking into at least 2 compartments.

8. The resin cleaning and sterilizing system of claim 1, wherein the second mesh basket includes a dividing member.

9. The resin cleaning and sterilizing system of claim 8, wherein the dividing member selectively divides the second mesh basket into at least two compartments.

10. A method of cleaning and sterilizing a smoking apparatus using a resin cleaning and sterilizing system, the resin cleaning and sterilizing system including a first ultrasonic cleaner having a first cavity configured to be filled with a cleaning solution and to receive a first mesh basket, and a second ultrasonic cleaner having a second cavity configured to be filled with a sterilizing solution and to receive a second mesh basket, the method comprising:
submerging the smoking apparatus into the cleaning solution of the first ultrasonic cleaner;
cleaning the smoking apparatus by activating the first ultrasonic cleaner for a first predetermined length of time;
removing the smoking apparatus from the cleaning solution after the first ultrasonic cleaner has been activated for the first predetermined length of time;
submerging the smoking apparatus into the sterilizing solution of the second ultrasonic cleaner;
sterilizing the smoking apparatus by activating the second ultrasonic cleaner for a second predetermined length of time; and
removing the smoking apparatus from the sterilizing solution after the second ultrasonic cleaner has been activated for the second predetermined length of time.

11. The method of claim 10, further comprising selecting at least one of a time for the first ultrasonic cleaner to be active, a temperature of the cleaning solution within the first cavity, and a frequency output by the first ultrasonic cleaner.

12. The method of claim 10, further comprising selecting at least one of a time for the second ultrasonic cleaner to be active, a temperature of the sterilizing solution within the second cavity, and a frequency output by the second ultrasonic cleaner.
